(19) United States
(12) Patent Application Publication
(10) Pub. No.: US 2004/0241144 A1
(43) Pub. Date: Dec. 2, 2004

(54) Cell compositions for use in the treatment of osteo-arthrosis, and methods for producing the same

(76) Inventors: Christian Kaps, Berlin (DE); Michael Sittinger, Gross-Ziethen (DE)

Correspondence Address: JONES DAY, 222 EAST 41ST ST, NEW YORK, NY, 10017, US

(21) Appl. No.: 10/486,956

(22) PCT Filed: Aug. 13, 2002
(86) PCT No.: PCT/EP02/09080

(30) Foreign Application Priority Data
Aug. 14, 2001 (DE) 101 39 783.6

(51) Int. Cl. A61K 45/00

(52) U.S. Cl. 424/93.7

(57) ABSTRACT

The present invention relates to the field of tissue engineering, in particular to the replacement of pathological tissue in joints (mainly bones and cartilage) and the treatment and the prevention of osteo-arthritic conditions in joints. For this purpose, the invention discloses methods for producing cell compositions, which comprise the provision of mesenchymal cells and synovial fluid, as well as the mixture thereof for obtaining a cell composition. Cell compositions are provided, which are used for the treatment of osteo-arthrosis and articular diseases or defects. Furthermore, the cell compositions are used for producing transplants. Finally, the present invention relates to methods for treating articular defects.

The present invention relates to the field of tissue engineering, in particular to the replacement of pathological tissue in joints (mainly bones and cartilage) and the treatment and the prevention of osteo-arthritic conditions in joints. For this purpose, the invention discloses methods for producing cell compositions, which include the provision of mesenchymal cells and synovial fluid, as well as the mixture thereof for obtaining a cell composition. Cell compositions are provided, which are used for the treatment of osteo-arthrosis and articular diseases or defects. Furthermore, the cell compositions are used for producing transplants. Finally, the present invention relates to methods for treating articular defects.

The osteo-arthrosis is the most frequent articular disease worldwide, the majority of all people in the age above sixty-five is affected thereof. Inevitably, from it results a very high clinical, health-political and economical relevance. In the course of said articular disease, which is primarily degenerative age-related, arise a step-wise focal destruction of the joint surface and a reactive mal-regulated regional growth of the adjacent and sub-chondral bone structures (osteophytes). The results are pains and limited function and motility of the affected joint. Systemic factors, which influence the genesis of an osteo-arthrosis, are the age, the gender, weight, acute osteoporosis, a familial preload and mechanical overload. Local factors are the specific joint form, mal-positions, traumas, as well as bio-mechanic factors, which affect the joint. Despite the underlying degenerative genesis, also in the osteo-arthrosis inflammable changes arise like a synovitis (inflammation of the inner joint skin), as well as a production of inflammation promotional biological messengers, for example of cytokines and growth factors (Rubin, J. Am. Osteopath. Assoc., 101, 2001, p. 2-5; van der Kraan and van den Berg, Curr. Opin. Nut. Metab. Care, 3, 2000, p. 205-211).

The proceeding changes are a defective regulation of the tissue homeostasis in the region of the heavily loaded cartilage structures and bone structures, that is there exists a dysbalance between degenerative and reparative methods. Thereby, the disease is the consequence of dysfunctions in the region of the complete joint including the bone, the musculature and the joint enervation, what finally leads to a mechanical overload and to a biochemically imported destruction of the affected joint.

It is furthermore important, that there is no healing of the disease osteo-arthrosis. Physio-therapeutic methods and analgetic, anti-inflammatory drugs (for example non-steoridal anti-rheumatics) are in many cases only insufficient symptomatic therapies. Known (conventional) orthopedic methods like debridement, joint-shaving, micro-fracture and drilling are also only insufficiently effective (see Fitzgibbons, T. C., AAOS Instructional Course Letters, Vol. 48, 1999, p. 243-248). In case of distinct degenerative changes, as final method frequently only the operative-reconstructive surgery with endo-prosthetic joint replacement remains, whereby the last mentioned method certainly is a very drastic method, which should be avoided as far as possible.

The tissue engineering offers promising new technologies by means of the possibility of a transplantation of functional active autologous cells, if necessary with the aid of shaping biomaterials. By means of such technologies, new tissue can be built up actively or can be grown (see Sittinger, M. et al., Biomaterials, 1994, p. 451-456; Redlich, A. et al., J. Mat. Sci. 10, 1999, p. 767-772). So, for example, new created tissue can be provided by means of genetic engineering or by means of the use of appropriate substrates and factors with immunosuppressive properties (see DE-A 196 32 404), so that no longer a rejection reaction against the transplant takes place or that said reaction is weakened.

The DE-C 44 31 598 describes a method for the manufacture of an implant from cell cultures, which includes the application of the cells onto a three-dimensional carrier structure and subsequent perfusion with a culture medium up to the at least partially formation of the intercellular matrix. Subsequently, said structure is implanted into the patient.

Likewise, the DE-C 43 06 661 describes the manufacture of coated dimensionally stable carrier structures, which are subsequently transplanted.

Finally, the DE-A 199 57 388 describes implantable substrates for the healing of cartilage and for the cartilage protection, which enable activating local cells, and therefore accelerating the healing method respectively the overgrowing of the affected region with cells. The substrates to be used are applied here onto the affected joint surface, preferably in the form of a paste, after the drilling of the bone, that is after creating of ducts between joint cavity and bone marrow cavity.

Therefore, the drawback of the methods, compositions and implants of the state of the art with regard to the healing of articular defects is that said methods, compositions and implants frequently require the opening of the joint respectively extensive mechanical manipulations at or within the joint. In particular this applies, if implants, which are adhered to carrier structures (and are therewith three-dimensional), have to be inserted into the joint. Consequently, the infection risk increases and the healing of the affected joint is made difficult or is made impossible. Furthermore, the conditions, which exist during the perfusion of an implant, are different from the physiological conditions, which exist within the joint.

Consequently, there is a strong need in the state of the art developing an alternative being as sparing as possible in order to treat osteo-arthritic articular defects. Furthermore, there is a need providing compositions for the treatment of osteo-arthrosis and related pathologies, which allow a regeneration of the affected joint or the joint region, which is as close to in vivo conditions and as efficient as possible. Furthermore, there is a need providing transplants, which were cultivated under conditions close to in vivo conditions, and which provide a high degree of biocompatibility. Finally, there is also a need in the state of the art providing compositions, which allow the treatment of the articular defect preferably with minimal invasive techniques.

As a result, it is an object of the present invention providing compositions, which provide a sparing, close to in vivo conditions and efficient treatment of degenerative articular diseases, in particular of osteo-arthrosis. Another object of the present invention is the providing of methods for the manufacture of the compositions according to the invention, as well as the providing of transplants, which are produced by using the compositions according to the invention. Finally, one object of the present invention is the providing of treating methods for osteo-arthrosis and similar degenerative articular diseases.

This and further objects are achieved by means of the methods and cell compositions according to the invention.

Therefore, the present invention relates to a method for the manufacture of a cell composition including the following steps:

a) providing of mesenchymal cells,

b) providing of synovial fluid,

c) mixing of synovial fluid and mesenchymal cells for the obtaining of a cell composition.

The providing of the mesenchymal cells, which is described in step a) includes in a preferred embodiment the use of freshly isolated cells for example from bone marrow, cartilage or blood. For example, the cells can be taken also as tissue respectively cartilage from a firstly unloaded region of a joint by means of cartilage biopsy. Then, subsequently, from said cartilage biopsy individual cartilage cells are isolated by means of enzymatic digestion. In a preferred embodiment of the present invention, before the providing the cells are cultivated in cell cultures and are proliferated. Particularly preferred is here the cultivation of the cells in suspension culture, so that the later mixture, in particular the preferred mixture of the mesenchymal cells and the synovial fluid in vitro (see step c)), can be achieved without problems and without applying mechanical methods. In a preferred embodiment of the present invention, the providing of the synovial fluid (step b)) relates to the use of frozen synovial fluid, which was defrosted before the use. In a preferred embodiment, the synovial fluid is taken before the mixing (step c)) with the mesenchymal cells from the joint, whereby an extraction of the synovial fluid directly before the mixing with the mesenchymal cells is in particular preferred.

The mixing of mesenchymal cells and synovial fluid can take place in arbitrary sequence. With respect to the cell number per volume unit (cell density), which exists after mixing, a density of 1 cell/mL to 40 million cells/mL (final cell density) is advantageous, preferred is a cell density of 2 million to 30 million cells/mL fluid, particularly preferred is a cell density of 5 million to 15 million cells/mL (final cell density).

In a preferred embodiment, the present invention also relates to a method, wherein the mesenchymal cells, which are provided in step a) are isolated from bone marrow, fat tissue, blood, spongy bone, cartilage or other mesenchymal tissues. In principle, all tissues can be used here, which contain the mesenchymal cells. The methods for the isolation of said cells are known to the one skilled in the art (see Haynesworth, S. E., Goshima, J., Goldberg, V. M., Caplan, A. I., Bone 13(1) (1992), p. 81-88; Haynesworth, S. E., Baber, M. A., Caplan, A. I., Bone 13 (1992), p. 69-80; Pittenger, M. F., Mackay, A. M., Beck, S. C., Jaiswal, R. K., Douglas, R., Mosca, J. D., Moorman, M. A., Simonetti, D. W., Craig, S., Marshak, D. R., Science 284 (1999), p. 43-147; Burmester, G. R., Menche, D., Merryman, P., Klein, M. Winchester, R., Arthritis Rheum. 26 (1983), p. 1187-1195; Sittinger, M., Bujia, J., Minuth, W. W., Hammer, C., Burmester, G. R., Biomaterials 15 (1994), p. 451-456; Sittinger, M., Reitzel, D., Dauner, M., Hierlemann, H., Hammer, C., Kastenbauer, E., Planck, H., Burmester, G. R., Bujia, J., J. Biomed. Mat. Res. 33 (1996), p. 57-63; as well as U.S. Pat. No. 5,486,359).

The present invention also relates to a method, wherein the mesenchymal cells, which are provided in step a), are mesenchymal precursor cells or are mesenchymal ancestral cells. The isolation and cultivation of human embryonic ancestral cells, which is described in the state of the art, in principle opens the possibility creating under appropriate cultivation conditions and development conditions from said omnipotent cells any body-innate cell form of the most different cell populations as for example cartilage cells, bone cells, skin cells, muscle cells, liver cells, kidney cells and neural cells. The high complexity of the relevant control cycles, which are relevant for said tissue-specific cell development, ethical reasons as well as the limited availability of said cells, however, can cause significant problems. The use of mesenchymal precursor cells according to the invention for the healing of bone defects and cartilage defects is advantageous, because said cells are already advanced in their development, and are determined with respect to their development potential regarding mesenchymal cell types.

Mesenchymal precursor cells in the meaning of the present invention also include mesenchymal ancestral cells. Mesenchymal precursor cells as well as mesenchymal ancestral cells provide a high reproduction capacity (Caplan, A. I., Clin. Plast. Surg. 21, 1994, p. 429-435) and are able under appropriate culture conditions respectively after appropriate manipulation, for example by means of genetic change, to develop directly to cells of mesenchymal tissue, that means to cartilage, bones, muscle, fat tissue and connective tissue. They can be gained from the blood, bone marrow and fat tissue of adult donators (Pittenger, M. F. et al., Science, 1999, p. 143-147), so that the ethically contended use of embryos, totipotent embryonic cells or embryonic tissue can be avoided in the context of the present invention. This ensures the medical/pharmaceutical applicability and producibility of cell compositions according to the invention. Furthermore, usually the known methods of the tissue engineering, which are known from the state of the art, are based on the reproduction of autologous cells, which subsequently are re-implanted into the patient, for example in the form of a fully developed transplant. Unfortunately, the proliferation potential of said cells is limited and a reproduction in vitro by means of many cell sequences reduces essentially the functional quality of the cells, what in turn makes these cells less suitable for the transplantation. Therefore, the use of mesenchymal precursor cells according to the invention, which are not subjected to the mentioned restrictions, is advantageous.

Furthermore, the present invention relates in a particular preferred embodiment to a method, wherein the mesenchymal cells, which are provided in step a), are autologous. “Autologous” in the meaning of the present invention means that cells are used, which were taken from the transplantation patient himself before the transplantation. This provides the considerable advantage—similar to the Eigen-blood-donation before a bigger operation, that for the transplantation or injection cells or cell compositions are used, which are genetically and immunologically adjusted perfectly to the acceptor (concerning the MHC-histocompatibility).

In a preferred embodiment, the present invention further relates to a method, wherein the mesenchymal cells, which are provided in step a), are heterologous. If no autologous cells are available for the transplantation, then also heterologous cell or cell compositions can be applied within the context of the invention. “Heterologous” means in this context, that mesenchymal cells can be used for the production thereof from an individual, which is different from the acceptor.

Furthermore, in a preferred embodiment, the invention relates to a method, wherein the mesenchymal cells, which are provided in step a), are cultivated in cell culture. Thereby, the cultivation of the mesenchymal cells takes place by using the cell culture techniques, which are known to the one skilled in the art, before the provision of the cells, advantageous is a culture period from 5 to 50 days, preferred are 10 to 40 days, in particular preferred is a period of 20 to 25 days.

The invention relates in a preferred embodiment to a method, wherein the synovial fluid is gained from a joint. Preferably, the synovial fluid is gained by means of puncture with a sterile needle or syringe directly from the joint. Thereby, the joint can be part of a living mammalian (including human beings). Furthermore, the synovial fluid, however, can be gained from dead mammalians respectively donators.

In another preferred embodiment, the present invention relates to a method for the manufacture of a cell composition, wherein the synovial fluid is autologous. “Autologous” in the meaning of the present invention means that synovial fluid is used, which was taken before the transplantation from the transplantation patient himself. This has—as already mentioned above with respect to the mesenchymal cells—the considerable advantage that cells respectively cell compositions can be used for the transplantation or for the injection, which are perfectly adjusted to the acceptor genetically and immunologically. This minimizes the risk of a rejection reaction of the acceptor respectively eliminates largely said reaction.

Furthermore, the invention relates to a method, wherein the synovial fluid which is provided in step b), is produced synthetically. The synthetic manufacture of synovial fluid means in the context of the present invention that a solution is produced, which is re-engineered to be similar to the natural synovial fluid, whereby in a preferred embodiment said solution is cell-free. In another preferred embodiment, the synthetic synovial fluid is protein-free, whereby a cell-free and protein-free synthetic synovial fluid is in particular preferred. The last-mentioned represents a synovial fluid, which, because it is essentially free from immunogens, is compatible with a multitude of donators, and therefore can be applied universally. Furthermore, the present invention relates to a method, wherein the synovial fluid, which is provided in step b), is modified chemically, physically, or biologically. “Chemical modification” in the context of the present invention is the treatment of the synovial fluid by means of predominantly chemical methods. Exemplified for chemical modification is ion-exchange chromatography, affinity chromatography, salting out, shaking out, fractional precipitation, treating with or supplying of chemicals, adjusting of the pH-value with acid or base, dialysis and reaction of the synovial fluid with chemicals. “Physical modification” in the context of the present invention means the treatment of the synovial fluid by means of predominantly physical methods. Exemplified for physical modifications are here centrifugation, heat/cooling treatment, boiling, cooling down etc. “Biologic modification” in the context of the present invention means the treatment of the synovial fluid by means of predominantly biological methods. Exemplified for biological modifications are here the modification of cells by genetic engineering, the supply/extraction of cells from the fluid and the treatment of the fluid with bacteria, virus, fungi or microorganisms or the metabolites thereof. Also the supply of biologic active substances or molecules is a biological modification in the context of the present invention. In particular preferred in the context of the present invention is a synovial fluid, in which the protein, which is present after the extraction (for example by means of precipitation and subsequent centrifugation) and/or the cells or cell parts (for example by means of centrifugation), which are present after the extraction, are removed, so that in step c) the mesenchymal cells can be mixed with “cleared” (that means largely cell-free and/or protein-free) synovial fluid.

In another preferred embodiment the present invention relates to a method, wherein the mesenchymal cells to be used are bipotent or pluripotent. Contrary to the omnipotent cells respectively cell-lines (synonym: totipotent cells or cell-lines, which are mentioned above, which have in a high degree embryonic character, and which can be differentiated into any desired tissue, the bipotent or pluripotent cells, which are preferably used in the context of the present invention, have already a certain—even though low—differentiation degree, and can be gained from adult donators. This leads to a better availability of such cells and to an extraction being trouble-freer. “Bipotent” in the meaning of the present invention means that the used mesenchymal (precursor) cells can differentiate into two different cell types, whereas “pluripotent” means that more than two cell types can arise by means of differentiation. The ethic concerns, which are discussed at present with respect to the use of embryonic tissue (the availability thereof nevertheless is restricted) or of embryonic cells in therapeutic methods respectively in the manufacture of drugs, step back when using bipotent or pluripotent cells. For this reason, the present invention is based on the use of bipotent or pluripotent mesenchymal (precursor) cells, which include in the meaning of the present invention bipotent or pluripotent mesenchymal ancestral cells (see above), for the tissue regeneration. The potential thereof for the proliferation and differentiation is principally likewise only little limited, whereby said cell type is of particular interest for the tissue engineering of cartilage and bones.

Furthermore, the invention relates in a preferred embodiment to a method, wherein mesenchymal cells, which are provided in step a), are treated with growth factors and/or differentiation factors, cytokines, extra-cellular matrix components or chemotactic factors. The differentiation behavior of the mesenchymal precursor cells can be influenced under defined culture conditions or also in vivo by means of the influence of different growth factors and differentiation factors as for example EGF, PDGF, IGF or FGF or by means of factors, which result from the TGF-β super-family. The cytokines, which are used within the scope of the present invention, comprise interleukins, EGF, HGF, PDGF, FGF as well as the TGF-family. Exemplified are the collagens as extracellular matrix components. In the context of the present invention, also chemotactic factors, as for example VEGF, SDF-1, MDC, MIP, “steel factor”, GM-CSF and interleukins can be used for the treatment of the mesenchymal precursor cells. Here, the terms “treating” or “treatment” have to be understood in a way that the mesenchymal cells are exposed to the above-mentioned substances respectively factors or are incubated in the presence thereof for a certain period or are mixed with said factors.

The invention also relates to a method, wherein the mesenchymal cells, which are provided in step a), are modified by genetic engineering. In a preferred embodiment, the modification by genetic engineering of the mesenchymal cells takes place by insertion of a plasmid, which for example permits the expression of a desired enzyme or structure protein, into the cells, which are as the case maybe in culture. However, it is also possible, using cells, whose chromosomes were modified, for example by means of chemical agents or integration vectors. In this manner, advantageous properties can be applied to the cells of the mesenchymal cell composition, which produce positive actions at the place of the later treatment (that means preferably in the affected joint). For example, such an action can be the over-expression of extra-cellular matrix proteins (collagens), which lead to an increased and accelerated settlement of further cells and cell organizations on the affected respectively surgically pre-treated joint area.

The invention also relates to a cell composition, obtainable according to one of the methods mentioned above.

The present invention also relates to the use of a cell composition obtainable according to one of the above-mentioned methods for the treatment of human and animal articular defects. Articular defects in the meaning of the present invention are pathological changes of a joint, which are caused by methods of inflammable and non-inflammable genesis.

The present invention also describes the use of a cell composition obtainable according to one of the above-mentioned methods for the injection into the joint space. In a preferred embodiment, thereby, the starting materials are autologous mesenchymal cells, which are inserted into an affected joint in autologous synovial fluid as “natural synovial fluid”. The application of the mesenchymal cells into the joint space or directly into the defect permits the settlement of cells, which are able to divide and which are able to mature within the defect region under physiologic conditions for the formation and regeneration of renewed cartilage or also bones.

Further, the invention relates to the use of a cell composition obtainable according to one of the above-mentioned methods for the injection into the joint defect. Besides the injection into the joint space (see above), owing to the circumstances, it can be advantageous for topologic reasons within the context of the present invention injecting the cell composition into the defect itself.

The invention also relates to the use of a cell composition obtainable according to one of the above-mentioned methods for the inter-operative treatment of articular defects. In the context of the present invention the term “inter-operative treatment” means that using the compositions according to the invention uses the period, which is between two joint operations, for the treatment of the articular defects.

Finally, the invention relates to the use of a cell composition obtainable according to one of the above-mentioned methods for the in vitro cultivation of mesenchymal tissue transplants. The cell composition, which is obtained in the steps a) to c), is used—besides the direct use for the injection into articular defects and in vivo treatments of said defects, which are resulting thereof—in a preferred embodiment for the in vitro cultivation, that is for the manufacture of transplants, which include mesenchymal cells. In a particular preferred embodiment, such transplants are cultivated by using three-dimensional supporting structures. Thereby, one starts from bio-compatible materials as for example polymer fleeces (including for example polyglycols or polylactides), plastic carriers or ceramic or mineral materials (for example hydroxyapatite), which serve as supporting structure, and which are settled or penetrated by mesenchymal cells. Preferably, this takes place in a chamber, which contains the cell composition according to the invention and the supporting structure, so that the supporting structure is surrounded by solution and the cells can adhere on said structure. In a particular preferred embodiment, said supporting structures are absorbable, so that after the adhesion of the mesenchymal cells on said structures and implantation into the articular defect, the supporting structure is successively resorbed. The transplant itself is obtained by cultivation of the mesencymal cell composition, which includes a synovial fluid, which was obtained from a joint or which is synthetic or is modified (see above), in presence of the supporting structure. Thereby, the supporting structure has in a preferred embodiment already the form, which the completed transplant should show. Methods for the manufacture of said transplants from cells and supporting structures or carrier structures are known to the one skilled in the art and are inter alia described in the WO 94/20151. The use of the cell composition according to the invention including synovial fluid (respectively modified or synthetic synovial fluid) has the advantage that the transplants are cultivated in a solution, which is very similar to the situation within the joint, so that a cultivation is possible, which is close to in vivo conditions. The latter results in stable transplants with a high degree of compatibility for the acceptor.

The term definitions, which are used in connection with the before-mentioned methods or uses, apply in the context of the present invention also for the cell compositions and treatment methods, which are listed in the following.

Furthermore, the invention relates to a cell composition including mesenchymal cells and synovial fluid, as well as to a cell composition, wherein the mesenchymal precursor cells are isolated from bone marrow, fat tissue, blood, spongy bone, cartilage or other mesenchymal tissues.

Furthermore, the invention relates in a preferred embodiment to a cell composition, wherein the mesenchymal cells are mesenchymal precursor cells. In the context of the present invention, “mesenchymal precursor cells” include also mesenchymal ancestral cells (see above).

The present invention also relates to a cell composition, wherein the mesenchymal cells are autologous, a cell composition, wherein the mesenchymal cells are heterologous, a cell composition, wherein the mesenchymal precursor cells are cultivated in cell culture, a cell composition, wherein the synovial fluid results from a joint, a cell composition, wherein the synovial fluid is autologous, as well as a cell composition, wherein the synovial fluid is produced synthetically.

Furthermore, the invention relates to a cell composition, wherein the synovial fluid is modified chemically, physically or biologically, a cell composition, wherein the mesenchymal cells are pluripotent, a cell composition, wherein the mesenchymal cells were treated with growth factors and/or differentiation factors, cytokines, extra-cellular matrix components or chemotactic factors, and to a cell composition, wherein the mesenchymal precursor cells were modified by genetic engineering.

Finally, the invention relates to a transplant, obtainable by the cultivation of a cell composition obtainable by means of one of the above-mentioned methods, on a carrier material. The preferred carrier materials, which form the carrier structure or the supporting structure (both terms are synonym in the context of the invention) were already mentioned above. The transplant is, as already described, produced by means of cultivation of the cell composition according to the invention in the presence of the carrier structure (also in solution or under perfusion).

The steps, substances and compositions, which are described in the treating methods mentioned below, are to be interpreted according to the definitions, which are already used above, unless not other defined.

The invention furthermore relates to a method for the treatment of human and animal articular defects, including the following steps:

(i) extraction of synovial fluid from a joint or production of synthetic synovial fluid,

(ii) mixing of the synovial fluid with mesenchymal cells,

(iii) injection of the mixture from (ii) into the joint.

The extraction of synovial fluid in step (i) from a joint is preferably carried out in a non-destructive manner by using a sterile needle or syringe. In the context of the present invention, the synovial fluid can either come from living donators or from dead mammalians (included human beings). The mixing of the synovial fluid, which was extracted, with mesenchymal cells in step (ii) can take place in any sequence. With regard to the cell number per volume unit (cell density), which is present after mixing, a density from 1 cell/mL to 40 million cells/mL (final cell density) is advantageous, preferred is a cell density from 2 million to 30 million cells/mL fluid, in particular preferred is a cell density from 5 million to 15 million cells/mL (final cell density). The injection of the mixture (step (iii)) into the joint of the acceptor should be carried out as sparing as possible and under sterile conditions. Within the scope of the present invention, the use of minimal-invasive techniques is preferred with regard to all treatment steps and treatment methods.

The invention also relates to a method, wherein step (i) is the production of synthetic synovial fluid.

The production of synthetic synovial fluid as well as the advantages thereof was already described in the context of the present invention (see above).

Further, the invention relates to a method, characterized by a chemical, physical or biological modification of the synovial fluid from step (i) between the steps (i) and (ii).

The different possibilities of the modification of the synovial fluid were already described above, herewith full reference is made to.

Finally, the invention relates to a method for the treatment of human and animal articular defects, wherein the mesenchymal cells are isolated from bone marrow, fat tissue, blood, spongy bone, cartilage or other mesenchymal tissues, a method, wherein the mesenchymal cells are autologous, a method, wherein the mesenchymals cells are heterologous, a method, wherein the mesenchymal cells are cultivated in cell cultures, a method, wherein the mesenchymal cells are mesenchymal precursor cells, as well as to a method, wherein the synovial fluid is autologous.

Finally, the present invention relates to a method, wherein the mesenchymal cells are bipotent or pluripotent, to a method, wherein the mesenchymal cells are treated with growth factors and/or differentiation factors, cytokines, extra-cellular matrix components or chemotactic factors, and to a method, wherein the mesenchymal cells are modified by genetic engineering.

The present invention should also be explained using the following embodiments. The embodiments are not regarded as being limiting, but are specifying the invention.

### EMBODIMENTS

### EXAMPLE 1

In order to provide a cell composition for the treatment of a joint surface, which is deformed arthritically, firstly autologous mesenchymal precursor cells are isolated from the bone marrow (see Haynesworth, S. E., Goshima, J, Goldberg, V. M., Caplan, A. I., Bone 13(1) (1992), p. 81-88; Haynesworth, S. E., Baber, M. A., Caplan, A. I., Bone 13 (1992), p. 69-80; Pittenger, M. F., Mackay, A. M., Beck, S. C., Jaiswal, R. K., Douglas, R., Mosca, J. D., Moorman, M. A., Simonetti, D. W., Craig, S., Marshak, D. R., Science 284 (1999), p. 43-147, as well as U.S. Pat. No. 5,486,359). The precursor cells are supplemented for 24 days under cell culture conditions with DME-medium (Biochrom KG, Berlin) and are cultivated with 10% autologous serum (DME-autologS).

By means of an aspiration needle, 5-10 mL synovial fluid are extracted from the diseased joint or from a sound joint, which subsequently is mixed with mesenchymal precursor cells, so that a cell concentration of 5 million cells/mL is achieved. For this, the precursor cells are detached from the culture surface by means of trypsin, and are treated with twice the volume of DME-autologS and are counted. In order to achieve a cell concentration of 5 million cells per mL synovial fluid, the respective volume of the precursor cell suspension is transferred into a centrifugal tube (15 mL) and is centrifuged at 300 g for 10 minutes at room temperature. The supernatant is discarded and the cell pellet is carefully mixed by means of a serologic pipette (5 mL) in the appropriate amount of synovial fluid by up- and down-pipetting. The cell composition, which is obtained in this manner, is directly injected per injection into the joint space. The application takes place by means of repeated supply of the cell composition in an interval of 2-3 weeks.

### EXAMPLE 2

In order to obtain a cell composition for the treatment of a circumscribed defect on a joint surface, at first autologous mesenchymal precursor cells are isolated from the bone marrow (see Haynesworth, S. E., Goshima, J, Goldberg, V. M., Caplan, A. I., Bone 13(1) (1992), p. 81-88; Haynesworth, S. E., Baber, M. A., Caplan, A. I., Bone 13 (1992), p. 69-80; Pittenger, M. F., Mackay, A. M., Beck, S. C., Jaiswal, R. K., Douglas, R., Mosca, J. D., Moorman, M. A., Simonetti, D. W., Craig, S., Marshak, D. R., Science 284 (1999), p. 43-147, as well as U.S. Pat. No. 5,486,359). The precursor cells are supplemented for 24 days under cell culture conditions with DME-medium and are cultivated with 10% autologous serum (DME-autologS).

5-10 mL synovial fluid are extracted from the diseased joint or from a sound joint by means of an aspiration needle, which is mixed with mesenchymal precursor cells, so that a cell concentration of 5 million cells/mL is achieved. For this, the precursor cells are detached from the culture surface by means of trypsin and are treated with twice the volume of DME-autologS and are counted. In order to achieve a cell concentration of 5 million cells per mL synovial fluid, the appropriate volume of the precursor cell suspension is transferred to a centrifugal tube (15 mL) and is centrifuged at 300 g for 10 minutes a room temperature. The supematant is discarded and the cell pellet is carefully mixed by means of a serologic pipette (5 mL) in the appropriate amount of synovial fluid by up- and down-pipetting. The cell composition, which is obtained in this manner, is injected directly into the defect.

### EXAMPLE 3

In order to obtain a cell composition for the treatment of an osteochondral defect in a joint, at first autologous mesenchymal precursor cells are extracted from the bone marrow (see Haynesworth, S. E., Goshima, J, Goldberg, V. M., Caplan, A. I., Bone 13(1) (1992), p. 81-88; Haynesworth, S. E., Baber, M. A., Caplan, A. I., Bone 13 (1992), p. 69-80; Pittenger, M. F., Mackay, A. M., Beck, S. C., Jaiswal, R. K., Douglas, R., Mosca, J. D., Moorman, M. A., Simonetti, D. W., Craig, S., Marshak, D. R., Science 284 (1999), p. 43-147, as well as U.S. Pat No. 5,486,359). The precursor cells are supplemented for 24 days under cell culture conditions with DME-medium and are cultivated with 10% autologous serum (DME-autologS).

By means of an aspiration needle, 5-10 mL synovial fluid are extracted from the diseased joint or from a sound joint and are mixed with mesenchymal precursor cells, so that a cell concentration of 10 million cells/mL is achieved. For this, the precursor cells are detached from the culture surface by means of trypsin and are treated with twice the volume of DME-autologS and are subsequently counted. In order to achieve a cell concentration of 5 million cells per mL synovial fluid, the appropriate volume of the precursor cell suspension is transferred into a 15 mL centrifugal tube, and is centrifuged at 300 g for 10 minutes at room temperature. The supernatant is discarded, and the cell pellet is carefully mixed by means of a serologic pipette (5 mL) in the appropriate amount of synovial fluid by means of up- and down-pipetting. The cell composition, which is obtained in this manner, is mixed with bone-inducing growth factors of the fibroblast growth factor super-family or the transforming growth factor-β (10 ng/mL final concentration) and is injected into the bony defect. After consolidation of the bony defect, a cell composition including mesenchymal precursor cells, as described in Example 1, is injected into the joint space in order to achieve a complete healing of the cartilage defect.

### EXAMPLE 4

For the treatment of a chondral defect of the joint, at first a cartilage biopsy is extracted from the unloaded region of the joint. By means of enzymatic digestion, cartilage cells are isolated from the cartilage biopsy (see Burmester, G. R., Menche, D., Merryman, P., Klein, M., Winchester, R., Arthritis Rheum. 26 (1983), p. 1187-1195; Sittinger, M., Bujia, H., Minuth, W. W., Hammer, C., Burmester, G. R., Biomaterials 15 (1994), p. 451-456; Sittinger, M., Reitzel, D., Dauner, M., Hierlemann, H., Hammer, C., Kastenbauer, E., Planck, H., Burmester, G. R., Bujia, J., J. Biomed. Mat. Res. 33 (1996), p. 57-63), supplemented in RPMI-medium (Biochrom KG, Berlin) and proliferated in the cell culture with 10% autologous serum (RPMI-autologS). By means of an aspiration needle 5-10 mL autologous synovial fluid are extracted from a sound joint, which is freed from cells by means of centrifugation at 300 g for 10 minutes at room temperature. The supernatant is mixed with cartilage cells, so that a cell concentration of 10 million cells/mL is achieved. For this, the cartilage cells are detached from the culture surface by means of trypsin and are treated with twice the volume of RPMI-autologS and are counted. In order to achieve a cell concentration of 10 million cells per mL synovial fluid, the appropriate volume of the cartilage cell suspension is transferred into a 15 mL centrifugal tube and is centrifuged at 300 g for 10 minutes at room temperature. The excess is discarded and the cell pellet is carefully mixed by means of a serologic pipette (5 mL) in the appropriate amount of synovial fluid by means of up- and down-pipetting. The cell composition, which is obtained in this manner, is injected in the meaning of an ACT (autologous chondrocyte transplantation) into the defect, which is over-sewed with an autologous bone skin.

1. Method for the manufacture of a cell composition including the following steps:
a) providing of mesenchymal cells,
b) providing of synovial fluid,
c) mixing of synovial fluid and mesenchymal cells for the obtaining of a cell composition.

2. Method as claimed in claim 1, wherein the mesenchymal cells, which are provided in step a), are isolated from bone marrow, fat tissue, blood, spongy bone, cartilage or other mesenchymal tissues.

3. Method as claimed in claim 1, wherein the mesenchymal cells, which are provided in step a), are mesenchymal precursor cells.

4. Method as claimed in claim 1, wherein the mesenchymal cells, which are provided in step a), are autologous.

5. Method as claimed in claim 1, wherein the mesenchymal cells, which are provided in step a), are heterologous.

6. Method as claimed in claim 1, wherein the mesenchymal cells, which are provided in step a), are cultivated in cell culture.

7. Method as claimed in claim 1, wherein the synovial fluid, which is provided in step b), is obtained from a joint.

8. Method as claimed in claim 1, wherein the synovial fluid, which is provided in step b), is autologous.

9. Method as claimed in claim 1, wherein the synovial fluid, which is provided in step b), is manufactured synthetically.

10. Method as claimed in claim 1, wherein the synovial fluid, which is provided in step b), is modified chemically, physically or biologically.

11. Method as claimed in claim 1, wherein the mesenchymal cells, which are provided in step a), are bipotent or pluripotent.

12. Method as claimed in claim 1, wherein the mesenchymal cells, which are provided in step a), are treated with a growth factor and/or differentiation factor, cytokine, extra-cellular matrix component or chemotactic factor.

13. Method as claimed in claim 1, wherein the mesenchymal cells, which are provided in step a), are modified by genetic engineering.

14. Cell composition obtainable according to the method as claimed in claim 1.

15. Method for the treatment of an articular defect in a human or non-human animal, comprising administering to said human or non-human animal a cell composition obtainable according to the method as claimed in claim 1.

16. Method as claimed in claim 15, wherein the cell composition is injected into a joint space of said human or non-human animal.

17. Method as claimed in claim 15, wherein the cell composition is injected into the articular defect of said human or non-human animal.

18. Method for the inter-operative treatment of an articular defect in a human or non-human animal, comprising administering inter-operatively to said human or non-human animal a cell composition obtainable according to the method as claimed in claim 1.

19. Method for obtaining a mesenchymal cell tissue for transplantation, comprising the in vitro cultivation of a cell composition obtainable according to the method as claimed in claim 1.

20. Cell composition including mesenchymal cells and synovial fluid.

21. Cell composition as claimed in claim 20, wherein the mesenchymal cells are isolated from bone marrow, fat tissue, blood, spongy bone, cartilage or other mesenchymal tissues.

22. Cell composition as claimed in claim 20, wherein the mesenchymal cells are mesenchymal precursor cells.

23. Cell composition as claimed in claim 20 or 22, wherein the mesenchymal cells are autologous.

24. Cell composition as claimed in claim 20 or 22, wherein the mesenchymal cells are heterologous.

25. Cell composition as claimed in claim 20 or 22, wherein the mesenchymal cells are cultivated in cell culture.

26. Cell composition as claimed in claim 20, wherein the synovial fluid originates from a joint.

27. Cell composition as claimed in claim 20, wherein the synovial fluid is autologous.

28. Cell composition as claimed in claim 20, wherein the synovial fluid is produced synthetically.

29. Cell composition as claimed in claim 20, wherein the synovial fluid is modified chemically, physically or biologically.

30. Cell composition as claimed in claim 20 or 22, wherein the mesenchymal cells are bipotent or pluripotent.

31. Cell composition as claimed in claim 20 or 22, wherein the mesenchymal cells are treated with a growth factor and/or differentiation factor, cytokine, extra-cellular matrix component or chemotactic factor.

32. Cell composition as claimed in claim 20 or 22, wherein the mesenchymal cells are modified by genetic engineering.

33. Transplant obtainable by means of the cultivation of a cell composition obtainable by the method as claimed in claim 1 or 3 on a carrier material.

34. Method for the treatment of an articular defect in a human or a non-human animal including the following steps:
(i) extraction of synovial fluid from a joint in said human or non-human animal or manufacture of synthetic synovial fluid,
(ii) mixing of the synovial fluid with mesenchymal cells,
(iii) injection of the mixture from (ii) into the joint.

35. Method as claimed in claim 34, wherein the step (i) is the manufacture of synthetic synovial fluid.

36. Method as claimed in claim 34, characterized by a chemical, physical or biological modification of the synovial fluid from step (i) between the steps (i) and (ii).

37. Method as claimed in claim 34, wherein the mesenchymal cells are isolated from bone marrow, fat tissue, blood, spongy bone, cartilage or other mesenchymal tissues.

38. Method as claimed in claim 34, wherein the mesenchymal cells are mesenchymal precursor cells.

39. Method as claimed in claim 34, wherein the mesenchymal cells are autologous.

40. Method as claimed in claim 34, wherein the mesenchymal cells are heterologous.

41. Method as claimed in claim 34, wherein the mesenchymal cells are cultivated in cell culture.

42. Method as claimed in claim 34, wherein the synovial fluid is autologous.

43. Method as claimed in claim 34, wherein the mesenchymal cells are bipotent or pluripotent.

44. Method as claimed in claim 34, wherein the mesenchymal cells are treated with a growth factor and/or differentiation factor, cytokine, extra-cellular matrix component or chemotactic factor.

45. Method as claimed in claim 34, wherein the mesenchymal cells are modified by genetic engineering.

46. Method as claimed in claim 11, wherein the mesenchymal cells are mesenchymal precursor cells.

47. Method as claimed in claim 12, wherein the mesenchymal cells are mesenchymal precursor cells.

48. Method as claimed in claim 13, wherein the mesenchymal cells are mesenchymal precursor cells.

49. Method for the treatment of an articular defect in a human or non-human animal, comprising administering into a joint of said human or non-human animal a composition comprising a mixture of mesenchymal cells and synovial fluid.
